(19) United States
(12) Patent Application Publication
(10) Pub. No.: US 2021/0401329 A1
(43) Pub. Date: Dec. 30, 2021

(54) METHODS AND DEVICES FOR EVALUATING HEARING SENSITIVITY OF INFANTS

(71) Applicant: Michael Mangini, Sycamore, IL (US)

(72) Inventor: Michael Mangini, Sycamore, IL (US)

(21) Appl. No.: 16/910,476

(22) Filed: Jun. 24, 2020

(51) Int. Cl. A61B 5/12; A61B 5/00; A61B 5/11

(52) U.S. Cl. CPC A61B 5/125; A61B 5/6846; A61B 5/0002; A61B 2503/04; A61B 5/121; A61B 2562/0247; A61B 5/11

(57) ABSTRACT

A method and device enable the detection of pressures induced by an infant's sucking actions on a mouthpiece. The method and device also enable the detection of head movements of the infant. The method and device transmit the data associated with the detected pressures and/or the data associated with the detected head movements to another device for evaluating the hearing sensitivity of the infant.

### FIELD OF THE DISCLOSURE

The present disclosure generally relates to techniques for evaluating the hearing sensitivity of infants, and more particularly, to determining possible hearing losses in the infants.

### BACKGROUND OF THE DISCLOSURE

Any missed or delayed identification of hearing loss (including partial hearing loss such as diminished hearing) in a child can have tremendous consequences in the child's later life (e.g., learning difficulties, special educational costs, lost job productivity, etc.). As such, it is critical that hearing loss is identified and remedied as early as possible to ensure the highest development of the child's communication and language skills. Yet, physicians often fail to detect hearing disorders in young infants. One of the reasons for this is that current diagnostic approaches are unsuitable for testing hearing responses of infants who are unable move their heads toward a sound stimulus. Furthermore, hearing screening of infants is not widely or readily available at the home. Accordingly, there remains a need to develop techniques to evaluate an infant's hearing ability with a high degree of accuracy for both physicians and parents alike.

### SUMMARY

According to some embodiments, the present disclosure provides a device that includes a mouthpiece with a portion adapted to be insertable into a mouth of an infant. The device also includes a pressure sensor configured to detect pressures induced by the infant's sucking actions on the mouthpiece. The device further includes a controller coupled to the pressure sensor and configured to transmit data associated with the detected pressures for use in assessing the infant's hearing.

For example, the controller is configured to transmit the data associated with the detected pressures wirelessly to a second device. As an example, the device includes a motion sensor configured to detect head movements of the infant. As such, the controller is coupled to the motion sensor and configured to transmit data associated with the detected head movements for use in assessing the infant's hearing. The motion sensor is also configured to detect sucking motions of the infant. As such, the controller is configured to transmit data associated with the detected sucking motions for use in assessing the infant's hearing. For example, the controller is configured to transmit the data associated with the detected pressures, the data associated with the detected head movements, and the data associated with the detected sucking motions wirelessly to the second device. As an example, the device includes a base portion coupled to the mouthpiece. The base portion includes the pressure sensor, the motion sensor, and the controller.

According to certain embodiments, the present disclosure provides a method performed in a first device. The method includes detecting, by a pressure sensor in the first device, pressures induced by an infant's sucking actions on a mouthpiece of the first device. The method also includes transmitting data associated with the detected pressures to a second device for use in assessing the infant's hearing.

For example, the method includes wirelessly transmitting the data associated with the detected pressures. As an example, the method includes detecting, by a motion sensor in the first device, head movements of the infant and transmitting data associated with the detected head movements to the second device for use in assessing the infant's hearing. For example, the method includes detecting, by the motion sensor in the first device, sucking motions of the infant and transmitting data associated with the sucking motions to the second device for use in assessing the infant's hearing. As an example, the method includes transmitting the data associated with the detected pressures, the data associated with the detected head movements, and the data associated with the detected sucking motions wirelessly to the second device.

According to some embodiments, the present disclosure provides a method performed in a second device. The method includes receiving, from a first device, data associated with detected pressures induced by an infant's sucking actions on a mouthpiece in the first device. The method also includes assessing the infant's hearing based on the data associated with the detected pressures.

For example, assessing the infant's hearing includes generating a hearing test signal with a modulated frequency that is based on the data associated with the detected pressures. As an example, assessing the infant's hearing includes generating a first hearing test signal with a frequency that is unmodulated and generating a second hearing test signal with a modulated frequency that is based on the data associated with the detected pressures. As such, assessing the infant's hearing includes determining whether a sucking behavior of the infant during the first hearing test signal is different from a sucking behavior of the infant during the second hearing test signal. For example, the method includes receiving, from the first device, data associated with detected head movements of the infant. As such, assessing the infant's hearing includes determining whether a head movement of the infant during the first hearing test signal is different from a head movement of the infant during the second hearing test signal. As an example, the method includes receiving, from the first device, data associated with detected sucking motions of the infant. As such, assessing the infant's hearing includes determining whether a sucking motion of the infant during the first hearing test signal is different from a sucking motion the infant during the second hearing test signal.

When the sucking behavior of the infant during the first hearing test signal is not different from the sucking behavior of the infant during the second hearing test signal, intensity levels of the first and second hearing test signals may be increased until the sucking behavior of the infant during the first hearing test signal differs from the sucking behavior of the infant during the second hearing test signal. On the other hand, when the sucking behavior of the infant during the first hearing test signal is different from the sucking behavior of the infant during the second hearing test signal, the intensity levels of the first and second hearing test signals may be decreased to determine a minimum intensity level that will cause the sucking behavior of the infant during the first hearing test signal to differ from the sucking behavior of the infant during the second hearing test signal. The intensity levels of the first and second hearing test signals may be generated based on an ambient noise level. Moreover, a calibration procedure may be performed to ensure that the generated intensity levels of the first and second hearing test signals conform to desired intensity levels.

### BRIEF DESCRIPTION OF THE DRAWINGS

The embodiments will be more readily understood in view of the following description when accompanied by the below figures and wherein like reference numerals represent like elements.

FIG. 1 is an assembly view of a pacifier device.

FIG. 2 is a bottom view of a housing portion of the pacifier device shown in FIG. 1.

FIG. 3 is a top view of a circuit board of the pacifier device shown in FIG. 1.

FIG. 4 is a bottom view of the circuit board of the pacifier device shown in FIG. 1.

FIG. 5 is a flowchart of a method for operating the pacifier device shown in FIG. 1.

FIG. 6 is a block diagram of a system for evaluating infant responses in a clinical setting.

FIG. 7 is a flowchart of a method for performing functions of the system shown in FIG. 6.

FIG. 8 is a flowchart of a method for evaluating infant responses with a standard audiometry method.

FIG. 9 is a flowchart of a method for evaluating infant responses in the clinical setting shown in FIG. 6.

FIG. 10 is a block diagram of a system for evaluating infant responses in a home setting.

FIG. 11 is a flowchart of a method for performing functions of the system shown in FIG. 10.

FIG. 12 is a flowchart of a method for device initialization and training in the home setting shown in FIG. 10.

FIG. 13 is a flowchart of a method for evaluating infant responses in the home setting shown in FIG. 10.

FIG. 14 is a flowchart of a method for operating a device for evaluating infant responses.

Corresponding reference characters indicate corresponding parts throughout the several views. The exemplifications set out herein illustrate exemplary embodiments of the disclosure and such exemplifications are not to be construed as limiting the scope of the disclosure in any manner.

### DETAILED DESCRIPTION

For the purposes of promoting an understanding of the principles of the present disclosure, reference is now made to the embodiments illustrated in the drawings, which are described below. The exemplary embodiments disclosed herein are not intended to be exhaustive or to limit the disclosure to the precise form disclosed in the following detailed description. Rather, these exemplary embodiments were chosen and described so that others skilled in the art may utilize their teachings.

The terms “couples,” “coupled,” and variations thereof are used to include both arrangements wherein two or more components are in direct physical contact and arrangements wherein the two or more components are not in direct contact with each other (e.g., the components are “coupled” via at least a third component or wirelessly), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

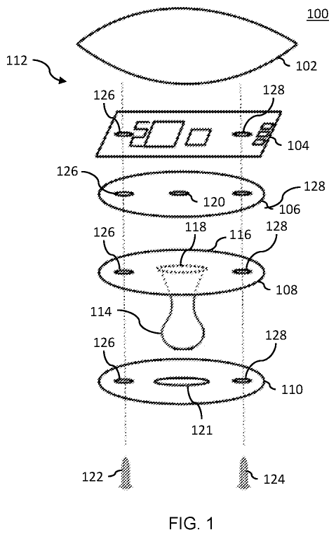

FIG. 1 shows an assembly view of a pacifier device 100 used by an infant. Pacifier device 100 is assembled using different components including a housing portion 102, a circuit board 104, a retention plate 106, a mouthpiece 108, and a mouthpiece retention ring 110.

Housing portion 102 provides the structure to hold components 104-110. Housing portion 102 is made from any suitable material such as translucent plastic. As an example, housing portion 102 is made from a molded plastic material. Housing portion 102 is sized to cover or fit around the lips of the infant. While FIG. 1 shows housing portion 102 as generally an oval shape, any shape or configuration may be contemplated. Circuit board 104 includes various electrical components, such as a plurality of sensors, configured to measure data associated with the infant. In some embodiments, circuit board 104 is a printed circuit board (PCB) with the various electrical components mounted thereto. Housing portion 102 and circuit board 104 form a base portion 112.

Retention plate 106 is used to provide an airtight/watertight seal between circuit board 104 and mouthpiece 108. Mouthpiece 108 has a portion (i.e., a nipple 114) that is adapted to be insertable into the mouth of the infant. For example, nipple 114 is a flexible piece that is inserted into the infant's mouth for the infant to suckle upon. In some embodiments, nipple 114 is attached to a plate 116 (e.g., using adhesives or other suitable means) via an opening 118 in plate 116. In certain embodiments, nipple 114 is an extension of mouthpiece 108. For example, mouthpiece 108 is formed from a continuous piece of flexible material (e.g., rubber, etc.) that also forms nipple 114. According to various embodiments, nipple 114 is made from any suitable material such as rubber, plastic, silicone, etc. There is also an air hole 120 in retention plate 106 that allows air to transfer from nipple 114 to a sensor (e.g., a pressure sensor) in circuit board 104. Accordingly, air hole 120 is aligned with the pressure sensor.

Mouthpiece retention ring 110 is used to retain nipple 114 and provide a cover for mouthpiece 108. Mouthpiece retention ring 110 also acts as a seal between mouthpiece 108 and the outside environment. Mouthpiece retention ring 110 includes an opening 121 though which nipple 114 can extend. According to various embodiments, the size and shape of retention elements 106, 110 conform to those of mouthpiece 108. The retention elements 106, 110 are made from any suitable material such as plastic or rubber.

Screws 122, 124 are used to fasten components 102-110 together and provide the airtight/watertight seal. Corresponding screw receptacles 126, 128 are in each of components 102-110. While the example of FIG. 1 shows the use of screws for fastening, any suitable fastening means (e.g., adhesives, rivets, press fit, etc.) to secure the components may be contemplated. In some embodiments, the airtight seal between retention plate 106 and mouthpiece 108 is created by using screws 122, 124 and/or adhesives.

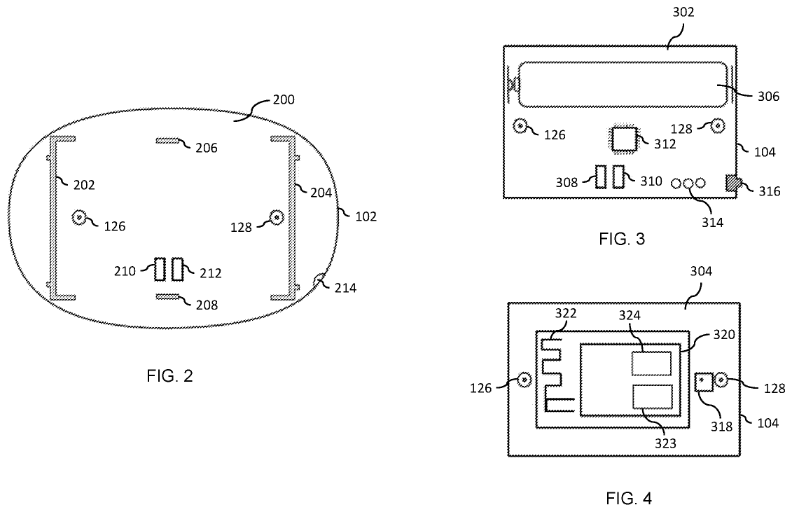

FIG. 2 shows a bottom 200 of housing portion 102. Raised ridges 202-208, conforming to the size and shape of circuit board 104, are disposed around the edges of bottom 200 to align and hold circuit board 104 in place. As an example, raised ridges 202-208 are made from any suitable material such as plastic. As described above, screw receptacles 126, 128 allow circuit board 104 to be fastened to housing portion 102 via screws 122, 124.

Two cutouts 210, 212 in housing portion 102 are located near raised ridge 208. Cutouts 210, 212 extend through a thickness of housing portion 102 and allow external leads (e.g., copper leads) to access circuit board 104 for charging purposes. There is also a pass-through hole 214 in housing portion 102 located near raised ridge 204. Pass-through hole 214 allows user interactions with circuit board 104 (e.g., pressing a button on circuit board 104).

FIGS. 3 and 4 show a first side 302 and a second side 304 of circuit board 104, respectively. First side 302 represents a top view of circuit board 104, while second side 304 represents a bottom view of circuit board 104. First side 302 of circuit board 104 is coupled to housing portion 102, specifically bottom 200 of housing portion 102. First side 302 of circuit board 104 includes a battery 306, electrical leads 308, 310, a motion sensor 312, one or more visual indicators 314, and a push button 316.

According to some embodiments, battery 306 is implemented as electrochemical cells in the form of a single battery (as shown in FIG. 3) or multiple batteries. For example, battery 306 is a rechargeable lithium ferrophosphate (LFP) battery. According to certain embodiments, the LFP type battery is desirable because it provides the necessary voltage over its lifecycle. Battery 306 is recharged via electrical leads 308, 310 that protrude or extend through cutouts 210, 212 in housing portion 102. Electrical leads 308, 310 are connected to battery 306 via electrical traces in circuit board 104. Depending on the application, battery 306 includes other types of rechargeable batteries. In some embodiments, battery 306 is disposable.

Motion sensor 312 is configured to detect head movements of the infant. For example, motion sensor 312 includes any combination of an accelerometer (used to detect changes in orientation with respect to gravity and linear acceleration in three dimensions), a three-axis gyroscope (used to detect changes in rotation), and/or a three-axis magnetometer (used to detect changes in direction with respect to a stable magnetic field). According to some embodiments, the accelerometer measures linear acceleration in the x, y, and z directions, and the gyroscope measures rotations in yaw, roll, and pitch. In some embodiments, motion sensor 312 is a single chip that includes the accelerometer, gyroscope, and magnetometer. In certain embodiments, separate accelerometer, gyroscope, and/or magnetometer devices are coupled together to form motion sensor 312.

Visual indicators 314, such as light-emitting diode (LED) lights, display the operational status of the components on circuit board 104 (e.g., a low battery status for battery 306, an activity status for motion sensor 312, status of communication link, etc.). Other types of visual indicators (e.g., a LED screen) may be contemplated. Visual indicators 314 are visible through housing portion 102 when housing portion 102 is made from a translucent material. In some embodiments, cutouts in housing portion 102 can be employed to expose visual indicators 314. Push button 316, which is accessible via pass-through hole 214, allows a user to interact with circuit board 104, such as pressing push button 316 for a system reset.

Second side 304 of circuit board 104 is coupled to mouthpiece 108 via retention plate 106. Second side 304 of circuit board 104 includes a pressure sensor 318, a controller 320, and antenna traces 322. Pressure sensor 318 is configured to detect pressures induced by the infant's sucking actions on mouthpiece 108. For example, as the infant bites on nipple 114 or changes the sucking rate on nipple 114, changes in the air pressure can be detected by pressure sensor 318 via air hole 120.

Controller 320 (e.g., a microcontroller, a microprocessor, a digital signal processor (DSP), an application specific integrated circuit (ASIC), etc.) is configured to receive data from sensors such as motion sensor 312 and/or pressure sensor 318. The received data are stored in a memory 323 of controller 320. Controller 320 is also configured to transmit the received data to an external device via a network. For example, controller 320 transmits data associated with the detected pressures and/or data associated with the detected head movements to the external device for use in assessing the infant's hearing.

Controller 320 includes a wireless communication unit 324. Wireless communication unit 324 is coupled to antenna traces 322 (e.g., copper traces) which enable the transmission of data (e.g., detected pressures, detected head movements) to the external device wirelessly. In some embodiments, sensors 312, 318 are surface mounted on circuit board 104 and coupled to controller 320 via electrical traces in circuit board 104. Sensors 312, 318 and controller 320 receive power from battery 306. In certain embodiments, circuit board 104 includes other types of sensors and more than one controller 320. According to various embodiments, base portion 112 includes sensors 312, 318 and controller 320.

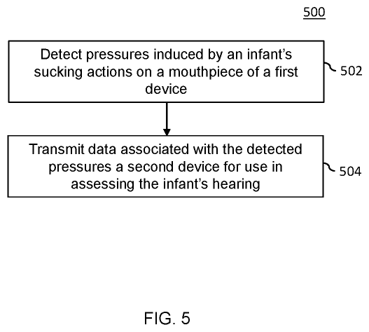

FIG. 5 shows a method 500 for operating a first device, such as pacifier device 100. As an example, method 500 is performed by a controller (e.g., 320) of the first device. At block 502, the first device detects pressures induced by an infant's sucking actions on a mouthpiece (e.g., 108) in the first device. The mouthpiece has a portion that is adapted to be insertable into a mouth of the infant. For example, the pressures are detected using a pressure sensor (e.g., 318) in the first device. At block 504, the first device transmits data associated with the detected pressures to a second device for use in assessing the infant's hearing. For example, the first device may transmit the data associated with the detected pressures wirelessly to the second device.

According to some embodiments, the first device detects head movements of the infant and transmits data associated with the detected head movements. According to certain embodiments, the first device detects sucking motions of the infant and transmits data associated with the detected sucking motions. For example, the head movements and sucking motions are detected using a motion sensor (e.g., 312) in the first device. In some embodiments, the first device transmits the data associated with the detected pressures, the data associated with the head movements, and the data associated with the detected sucking motions wirelessly to the second device.

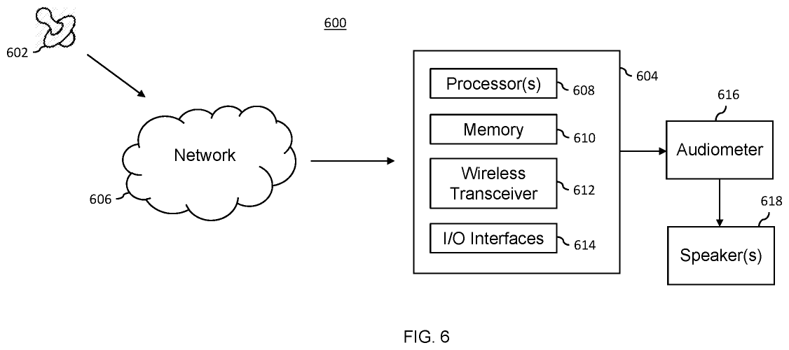

FIG. 6 shows a block diagram of a system 600 for evaluating data associated with an infant's hearing. In system 600, a first device 602 communicates with a second device 604 via a suitable communication network 606, such as a local area network (e.g., Wi-Fi), a wide area network (e.g., the Internet), a personal area network (e.g., Bluetooth), etc. As an example, first device 602 is pacifier device 100 of FIG. 1. First device 602 is configured to measure data associated with the infant (e.g., detected pressures induced by the infant's sucking actions, head movements of the infant, sucking motions of the infant) and transmit the data to be received by second device 604.

In FIG. 6, second device 604 is in the form of a clinical modulator used by an audiologist or other specialists in a clinical setting (e.g., hospitals, private clinics, etc.) for assessing the infant's hearing. For example, clinical modulator 604 includes any computing device with one or more processors 608 (e.g., microprocessors, microcontrollers, DSPs, ASICs, etc.), a memory 610 (e.g., random access memory (RAM), read-only memory (ROM), flash memory, etc.), a wireless transceiver 612, and input/output (I/O) interfaces 614 (e.g., displays, touch buttons, LED lights, etc.). In some embodiments, clinical modulator 604 is in the form of a tablet computer.

In the clinical setting, the infant is placed in a sound isolation booth. The sound isolation booth is constructed to keep background or ambient noise below a certain level (e.g., below ANSI 3.1 standards). Clinical modulator 604 is connected to an audiometer 616 which in turn is connected to one or more speakers 618 located in the sound isolation booth. The connection between clinical modulator 604, audiometer 616, and speakers 618 can be implemented using any suitable wired or wireless links.

Clinical modulator 604 receives data associated with the detected pressures (e.g., from first device 602) and generates one or more hearing test signals to present to the infant in the sound isolation booth via speakers 618. In some embodiments, a hearing test signal is a sine wave with a modulated frequency that is based on the data associated with the detected pressures. The purpose is to determine whether a hearing loss is present in the infant by examining the sucking behavior of the infant when the hearing test signal alternates between an unmodulated steady frequency tone and a frequency tone that is modulated by the detected pressures. Research has shown that infants as young as 2 months old can change their sucking behaviors in response to hearing a tone that is modulated by their sucking behaviors. As such, clinical modulator 604 generates a plot of the pressure data, sucking amplitude, sucking phase, and sucking frequency for the audiologist to examine (e.g., via I/O interfaces 614). In this manner, the audiologist then determines if the sucking behavior of the infant changed with the changes in the modulation of the hearing test signal. When the sucking behavior of the infant changes between the unmodulated and modulated hearing test signals, this indicates to the audiologist that the infant heard and responded to the tone changes. On the other hand, a lack of such change in the sucking behavior is evidence that the infant may be suffering from a hearing loss. While clinical modulator 604 has been described above for use in the field of clinical audiology, other applications in other fields or research may be contemplated.

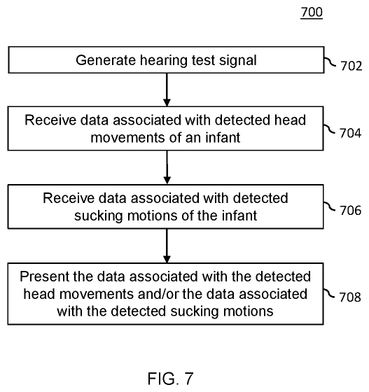

FIG. 7 shows a method 700 for performing the functions of system 600. At block 702, a hearing test signal is generated. For example, an audiologist selects or adjusts test frequencies for the hearing test signal using clinical modulator 604. At block 704, data associated with the detected head movements of the infant are received. For example, clinical modulator 604 receives the data associated with the detected head movements from first device 602. At block 706, data associated with the detected sucking motions of the infant are received. For example, clinical modulator 604 receives the data associated with the detected sucking motions from first device 602. At block 708, the data associated with the detected head movements and/or the data associated with the detected sucking motions are presented. For example, clinical modulator 604 presents the data associated with the detected head movements and/or the data associated with the detected sucking motions as information for the audiologist to consider when determining whether the infant has any hearing loss.

The current standard for testing hearing loss in infants (e.g., between 6-24 months old) is the Visual Reinforcement Audiometry (VRA) method. The VRA method is based on detecting head turns made by an infant to determine if the infant perceived and responded to a sound stimulus. For example, sounds on a particular side (either left or right) are paired with an upcoming interesting visual stimulus (e.g., a moving toy or a short-animated sequence on a display). During the learning phase, the infant learns to associate the sounds with the upcoming visual stimulus. A turning of the infant's head in the direction of the sounds that precedes the visual stimulus is evidence that the infant was able to hear the sounds.

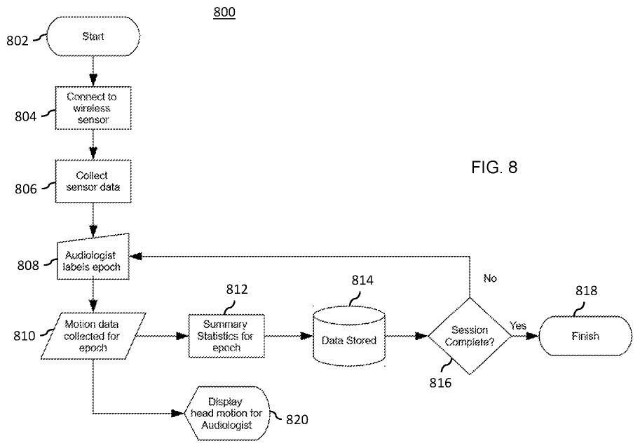

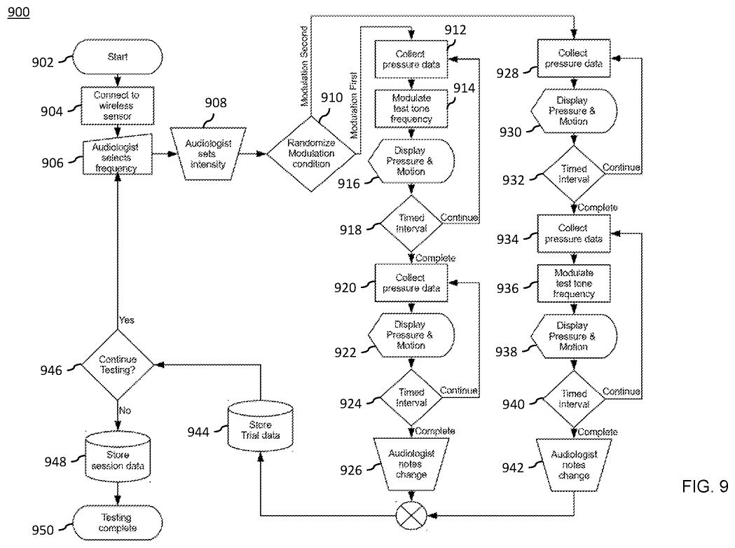

FIG. 8 shows a method 800 for evaluating infant responses with the VRA method. A hearing test session begins at block 802. As an example, the hearing test session includes one or more test epochs. At block 804, connections to one or more sensors (e.g., motion sensors, pressure sensors) are established. In some embodiments, the one or more sensors are configured as wireless sensors. At block 806, data from the one or more sensors are collected. At block 808, an audiologist labels (e.g., initiates) a test epoch. For example, the test epoch includes presenting audio tones to a left side of an infant. At block 810, upon initiating the test epoch, motion data from the infant (e.g., head motions of the infant) are collected as received from the one or more sensors. At block 812, the motion data for the test epoch are summarized (e.g., processed for statistical information) and then stored at block 814.

At block 816, if all the test epochs in the hearing test session are completed, the hearing test session ends at block 818. If other test epochs need to be performed, the audiologist initiates additional test epochs at block 808. The motion data collected from the infant are displayed for the audiologist at block 820.

FIG. 9 shows a method 900 for evaluating infant responses in a clinical setting, such as the clinical setting shown in FIG. 6. To begin, a testing session is initiated at block 902. For example, the testing session includes one or more trials to test the hearing ability of an infant. At block 904, connections are established to one or more sensors. For example, a connection to first device 602 is established by clinical modulator 604 to access the sensors of first device 602 (e.g., pressure sensor, motion sensor). For a given trial, a clinical tester (e.g., an audiologist) selects one or more parameters for a hearing test signal. At block 906, the clinical tester selects a frequency for the hearing test signal. At block 908, the clinical tester selects an intensity level for the hearing test signal. For example, I/O interfaces 614 of clinical modulator 604 includes buttons that allow the clinical tester to select a test frequency for the hearing test signal (e.g., 0.25, 0.5, 0.75, 1, 1.5, 2, 3, 4, 6, 8 kHz). As an example, the clinical tester can also increase or decrease the intensity level of hearing test signal in increments (e.g., by 5 dB) to determine a threshold measure. Based on the frequency and intensity level selected by the clinical tester, clinical modulator 604 generates the hearing test signal.

At block 910, clinical modulator 604 modulates the hearing test signal. For example, clinical modulator 604 modulates the hearing test signal by some fraction (e.g., ¼, ⅓, etc.) of an octave above or below the selected test frequency. According to various embodiments, clinical modulator 604 sends the hearing test signal to audiometer 616 for presentation to the infant.

The modulation of the hearing test signal at block 910 may be randomized with one of a first modulation condition or a second modulation condition. With the first modulation condition, clinical modulator 604 collects pressure data from first device 602 at block 912. For example, the pressure data indicate pressures induced by the infant's sucking actions on first device 602 (e.g., pacifier device 100). At block 914, clinical modulator 604 modulates the frequency of the hearing test signal. For example, the frequency of the hearing test signal is modulated based on the pressure data. At block 916, clinical modulator 604 displays the pressure data along with motion data (e.g., via I/O interfaces 614) for the clinical tester to view/analyze. In some embodiments, the motion data indicate sucking motions of the infant as detected by first device 602. For example, the sucking motions relate to head rotation data of the infant. As an example, the sucking motions relate to head orientation data of the infant. In various embodiments, the motions of the infant's jaw, lips, mouth, tongue, and even the pressures pulling first device 602 toward the infant's face all combine to create small measurable accelerations in the x, y, and z directions. This data is used as a secondary measure to encode the infant's tugging on first device 602.

Blocks 912-916 are executed for a first timed interval. At block 918, if the first timed interval is completed, clinical modulator 604 again collects pressure data from first device 602 at block 920. At block 922, clinical modulator 604 again displays the pressure data and motion data for the clinical tester to view/analyze. Blocks 920-922 are executed for a second timed interval. At block 924, if the second timed interval is completed, the clinical tester notes any changes in the pressure data and/or motion data at block 926. For example, the clinical tester determines if the sucking behavior of the infant has changed with changes in the modulation of the hearing test signal.

With the second modulation condition, clinical modulator 604 collects pressure data from first device 602 at block 928. At block 930, clinical modulator 604 displays the pressure data along with motion data (e.g., head rotation data, head orientation data, etc.) for the clinical tester to view/analyze. Blocks 928-930 are executed for a first timed interval. At block 932, if the first timed interval is completed, clinical modulator 604 again collects the pressure data from first device 602 at block 934. At block 936, clinical modulator 604 modulates the frequency of the hearing test signal (e.g., based on the pressure data and/or motion data). At block 938, clinical modulator 604 again displays the pressure data and motion data for the clinical tester to view/analyze. Blocks 934-938 are executed for a second timed interval. At block 940, if the second timed interval is completed, the clinical tester notes any changes in the pressure data and/or motion data at block 942.

At block 944, clinical modulator 604 saves all trial data for the given trial (e.g., in memory 610). At block 946, if additional trials for the testing session are needed, method 900 returns to block 906 begin another trial. If the testing session is completed, clinical modulator 604 saves all session data at block 948 (e.g., in memory 610) and the testing session ends at block 950. In some embodiments, the trial data and/or session data are saved in an external database coupled to clinical modulator 604 and/or in a third-party database.

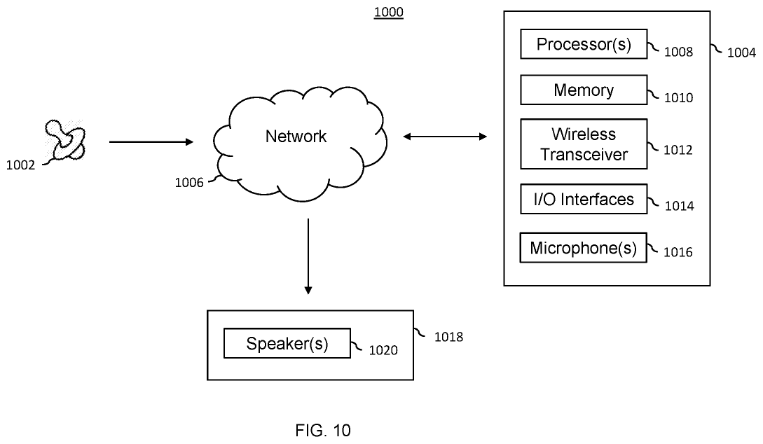

FIG. 10 shows a block diagram of a system 1000 for evaluating data associated with an infant's hearing. Similar to system 600, a first device 1002 communicates with a second device 1004 via a communication network 1006 in system 1000. Communication network 1006 may include any suitable local area network, wide area network, personal area network, etc. As an example, first device 1002 is pacifier device 100 of FIG. 1. First device 1002 is configured to measure data associated with the infant (e.g., detected pressures induced by the infant's sucking actions, head movements of the infant, sucking motions of the infant) and transmit the data to be received by second device 1004.

In FIG. 10, second device 1004 is in the form of a home testing device used by parents or caretakers of the infant in a home setting for assessing the infant's hearing ability. According to certain embodiments, home testing device 1004 includes any computing device with one or more processors 1008 (e.g., microprocessors, microcontrollers, etc.), a memory 1010 (e.g., RAM, ROM, etc.), a wireless transceiver 1012, I/O interfaces 1014 (e.g., displays, touch buttons, etc.), and one or more microphones 1016. In some embodiments, home testing device 1004 is enclosed in a toy (e.g., a stuffed animal) that is placed in a location where the infant will be sitting or lying.

Similar to clinical modulator 604, home testing device 1004 receives data associated with the detected pressures (e.g., from device 1002) and generates one or more hearing test signals to present to the infant. However, in the home setting, there are no trained audiologists to view or analyze the results of the hearing test. There also does not exist any sound isolation booth with calibrated speakers. Further, the amount of testing time and data needed for a reliable home test is much greater than in a clinical setting. As such, home testing device 1004 is configured to perform additional operations as described below.

According to some embodiments, home testing device 1004 is configured to work with a home audio system 1018 in the home (e.g., a home entertainment system, a smart speaker, etc.). Home audio system 1018 includes one or more speakers 1020. Prior to a hearing test, home testing device 1004 establishes a connection to speakers 1020 of home audio system 1018 for calibration purposes. Home testing device 1004 generates a series of sound stimuli (e.g., wideband audio noise, frequency sweeps), which are measured by microphones 1016 in home testing device 1004. This is done to ensure that the intensity levels of generated hearing test signals are consistent or conform to the desired intensity levels. In certain embodiments, instead of using speakers 1020 in home audio system 1018, home testing device 1004 includes its own remote speakers.

For all testing sessions, microphones 1016 in home testing device 1004 record all acoustic energy. This is done to obtain a continuous estimate of the background or ambient noise levels, which will determine a minimum allowable intensity for the hearing test signals. For example, in many homes, ambient noise can reach 60 dB sound pressure level (SPL) or greater. As such, testing sessions that fail during high ambient noise periods will not be considered valid.

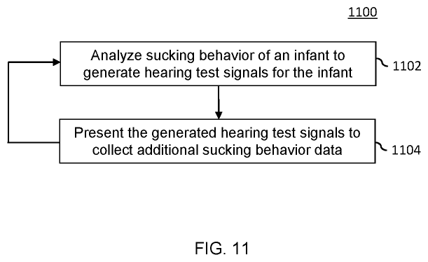

FIG. 11 shows a method 1100 for performing the functions of system 1000. At block 1102, the sucking behavior of an infant is analyzed to generate hearing test signals for the infant. For example, home testing device 1004 analyzes the sucking behavior of the infant at every transition from unmodulated to modulated test conditions to determine if there is evidence of a change in sucking behavior that is concurrent with the changes in the modulation of the hearing test signals. At block 1104, the generated hearing test signals are presented to the infant to collect additional sucking behavior data from the infant. For example, the additional sucking behavior data are again analyzed at block 1102.

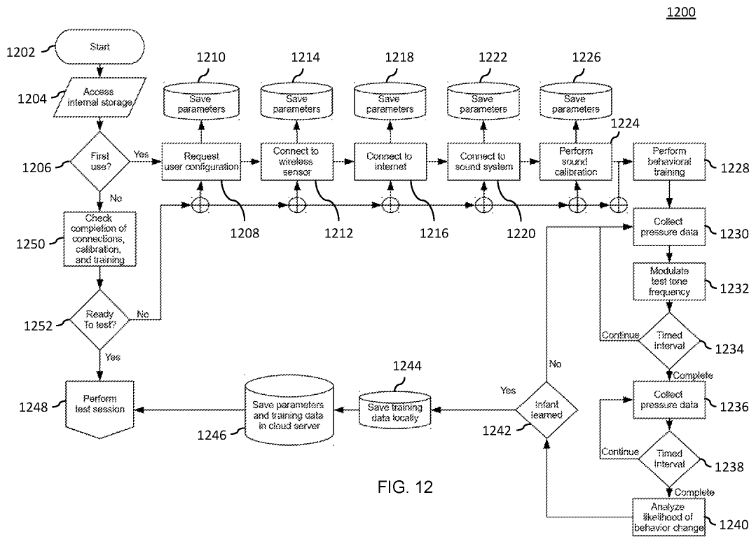

FIG. 12 shows a method 1200 for initializing home testing device 1004 and performing initial training sessions. In various embodiments, home testing device 1004 is programmed to perform a behavioral training session during which home testing device 1004 will generate hearing test signals, present these test signals to an infant, and collect feedback input from the parents. The behavioral training session is initiated at block 1202. At block 1204, home testing device 1004 accesses its internal storage files (e.g., saved in memory 1010).

At block 1206, it is determined whether home testing device 1004 is being used for the first time. If a first use is determined, home testing device 1004 requests user configurations at block 1208 with user configuration parameters being saved at block 1210. Next, home test device 1004 connects to one or more sensors (e.g., sensors of first device 1002) at block 1212 with sensor connection parameters being saved at block 1214. Home testing device 1004 also connects to a network (e.g., the Internet) at block 1216 with network connection parameters being saved at block 1218. Home testing device 1004 then connects to a sound system (e.g., home audio system 1018) at block 1220 with sound system connection parameters being saved at block 1222. Home testing device 1004 performs sound calibration at block 1224 with sound calibration parameters being saved at block 1226. In various embodiments, the parameters saved at blocks 1210, 1214, 1218, 1222, 1226 are stored in memory 1010 of home testing device 1004 or another suitable database.

Behavior training begins at block 1228. Specifically, home testing device 1004 generates hearing test signals for the infant in which the intensity level of the hearing test signals will begin at a moderately loud level (e.g., 60-70 dB) and the frequency of the hearing test signals will be modulated. At block 1230, home testing device 1004 collects pressure data from first device 1002. At block 1232, home testing device 1004 modulates the frequency of the hearing test signals. Blocks 1230-1232 are executed for a first timed interval. At block 1234, if the first timed interval is completed, home testing device 1004 again collects the pressure data from first device 1002 at block 1236 for a second timed interval.

At block 1238, if the second timed interval is completed, the collected pressure data are analyzed at block 1240 for the likelihood of behavioral changes. In particular, a series of test frequencies at the moderately loud level is presented to allow the infant to learn that his/her sucking behavior modulates the perceived pitch of the tone presented regardless of the particular test frequency. The parents are prompted by home testing device 1004 to indicate whether they believe the infant was aware of his/her sucking behavior modulating the tone. If the parents indicate that the infant did not appear to be aware of his/her “control” of the sound stimulus, then the modulation of the hearing test signals at the moderately loud level would repeat. Thus, if the infant has not learned, the behavioral training session repeats blocks 1230-1240. The unmodulated and modulated test conditions will continue at the moderately loud level until there is evidence of changes in the sucking behavior of the infant that is concurrent with changes in the modulation of the hearing test signals. If the infant has learned, all training data from the behavioral training session are saved locally at block 1244 (e.g., in memory 1010). At block 1246, the training data are saved to an external storage (e.g., a cloud server storage). Afterward, a testing session begins at block 1248. In other words, when the parents indicate that the infant appeared to be aware of his/her “control,” the testing session will commence.

If it is determined that home testing device 1004 is not being used for the first time, a check is performed on the completion of any device connection, device calibration, and/or behavioral training session. If any one of these is not completed, then blocks 1208-1246 are executed. Otherwise, the test session begins at block 1248.

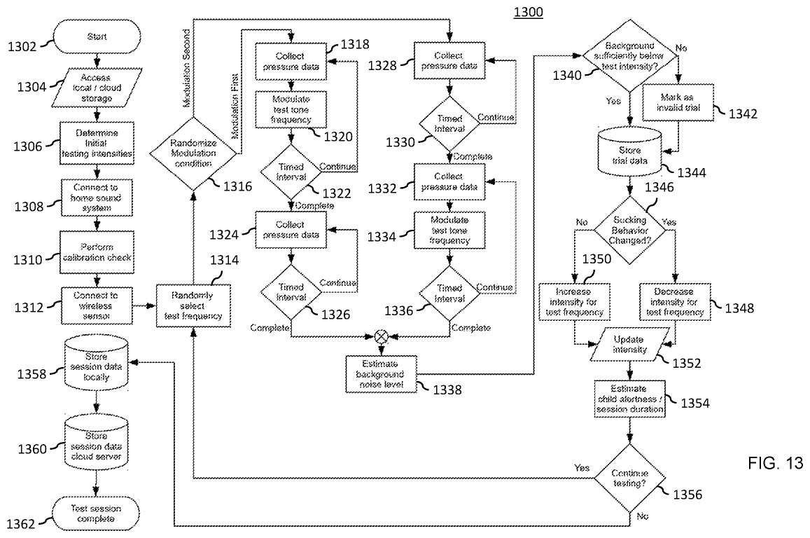

FIG. 13 shows a method 1300 for evaluating infant responses in a home setting, such as the home setting shown in FIG. 10. To begin, a testing session is initiated at block 1302. For example, the testing session includes one or more trials to test the hearing ability of an infant. At block 1304, home testing device 1004 accesses its internal storage files (e.g., via either local storage or cloud storage). At block 1306, home testing device 1004 determines initial testing intensities. At block 1308, home testing device 1004 connects to a home sound system (e.g., home audio system 1018). At block 1310, home testing device 1004 performs calibration checks. At block 1312, home testing device 1004 connects to one or more sensors (e.g., sensors of first device 1002). At block 1314, home testing device 1004 randomly selects a frequency to generate a hearing test signal.

At block 1316, home testing device 1004 modulates the hearing test signal. For a given trial, the modulation of the hearing test signal may be randomized with one of a first modulation condition or a second modulation condition. With the first modulation condition, home testing device 1004 collects pressure data from first device 1002 at block 1318. For example, the pressure data indicate pressures induced by the infant's sucking actions on first device 1002. At block 1320, home testing device 1004 modulates the frequency of the hearing test signal. For example, the frequency of the hearing test signal is modulated based on the pressure data. Blocks 1318-1320 are executed for a first timed interval. At block 1322, if the first timed interval is completed, home testing device 1004 again collects pressure data from first device 1002 at block 1324 until a second timed interval is completed at block 1326.

With the second modulation condition, home testing device 1004 collects pressure data from first device 1002 at block 1328. The pressure data are collected for a first timed interval. At block 1330, if the first timed interval is completed, home testing device 1004 again collects pressure data at block 1332. At block 1334, home testing device 1004 modulates the frequency of the hearing test signal. Blocks 1332-1334 are executed for a second timed interval until completion at block 1336.

At block 1338, the background noise level is estimated. At block 1340, if the background noise level is sufficiently below the intensity level of the hearing test signal, the given trial is marked as valid. Otherwise, the given trial is marked as invalid at block 1342. In any event, all trial data for the given trail are saved (e.g., in memory 1010) at block 1344.

At block 1346, a determination is made on whether the infant's sucking behavior has changed. Initially, the frequency of the hearing test signal will be presented at an intensity level below the moderately loud level (e.g., 10 dB) from the initial behavioral training session. If evidence indicates that the infant has perceived this lower intensity stimulus, then the hearing test signal will decrease in intensity (e.g., in steps of 10 dB) at block 1348. On the other hand, if evidence indicates that the infant did not change his/her sucking behavior concurrent with the modulation of the hearing test signal, then the intensity level of the hearing test signal will increase (e.g., in steps of 5 dB) at block 1350. The intensity level is then updated at block 1352.

At block 1354, the infant's alertness and/or testing session duration is estimated. At block 1356, if additional trials for the testing session are needed, method 1300 returns to block 1314 begin another trial. Home testing device 1004 will alternate the frequency of the hearing test signal between trials to minimize any adaptation to the sound stimuli on the part of the infant. If the testing session is completed, home testing device 1004 saves all session data locally at block 1358 (e.g., in memory 1010) as well as externally at block 1360 (e.g., cloud server storage). Afterward, the testing session ends at block 1362. In some embodiments, data received, logged, and/or analyzed by home testing device 1004 are sent (e.g., directly or via a cloud server) to an audiologist for further evaluation when desired or needed.

Unlike standard methods, the test frequencies alternate randomly between trials. Home testing device 1004 stores all the intensities for every test frequency, as well as the pressure and/or motion data recorded during the presentation interval. In some embodiments, to reduce the amount of data stored in home testing device 1004, rather than having the complete pressure and/or motion data streams, some statistical summaries of this data, or even single estimates of difference for the modulated vs. unmodulated intervals are stored for each trial. In certain embodiments, home testing device 1004 presents a hearing test signal at an intensity level appropriate for that test frequency based on the last presented intensity level and whether the evidence indicated that the infant did or did not perceive the modulation changes during the test.

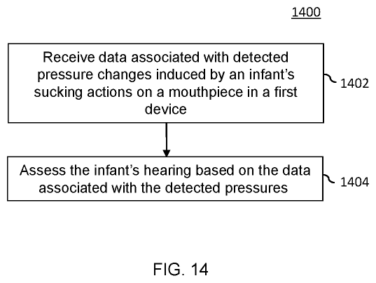

FIG. 14 shows a method 1400 for operating a second device, such as clinical modulator 604 and/or home testing device 1004. Method 1400 is performed by a processor (e.g., 608, 1008) of the second device. At block 1402, the second device receives data associated with detected pressures induced by an infant's sucking actions on a mouthpiece in a first device (e.g., 602, 1002). At block 1404, the second device assesses the infant's hearing based on the data associated with the detected pressures.

In some embodiments, to assess the infant's hearing, the second device generates a hearing test signal with a modulated frequency that is based on the data associated with the detected pressures. In certain embodiments, to assess the infant's hearing, the second device generates a first hearing test signal with a frequency that is unmodulated, and a second hearing test signal with a modulate frequency that is based on the data associated with the detected pressures. The second device then determines whether a sucking behavior of the infant during the first hearing test signal is different from a sucking behavior of the infant during the second hearing test signal (e.g., whether the sucking behavior is concurrent with changes in the modulation of the frequency in the first and second hearing test signals).

When the sucking behavior of the infant during the first hearing test signal is not different from the sucking behavior of the infant during the second hearing test signal, the second device increases (e.g., repeatedly) the intensity levels of the first and second hearing test signals until the sucking behavior of the infant during the first hearing test signal differs from the sucking behavior of the infant during the second hearing test signal. On the other hand, when the sucking behavior of the infant during the first hearing test signal is different from the sucking behavior of the infant during the second hearing test signal, the second device decreases (e.g., repeatedly) the intensity levels of the first and second hearing test signals to determine a minimum intensity level that will cause the sucking behavior of the infant during the first hearing test signal to differ from the sucking behavior of the infant during the second hearing test signal. In some embodiments, the intensity levels of the first and second hearing test signals are generated based on an ambient noise level. For example, the second device generates the first and second hearing test signals at intensity levels that are above an allowable intensity level determined by analyzing the ambient noise levels over a period of time. As an example, a calibration procedure is performed by the second device to ensure that the generated intensity levels of the first and second hearing test signals conform to desired intensity levels.

In some embodiments, the second device receives data associated with detected head movements of the infant. The second device assesses the infant's hearing by determining whether a head movement of the infant during the first hearing test signal is different from a head movement of the infant during the second hearing test signal (e.g., whether the detected head movements are concurrent with the changes in the modulation of the frequency in the first and second hearing test signals).

In certain embodiments, the second device receives data associated with detected sucking motions of the infant. The second device assesses the infant's hearing by determining whether a sucking motion of the infant during the first hearing test signal is different from a sucking motion of the infant during the second hearing test signal (e.g., whether the detected sucking motions are concurrent with the changes in the modulation of the frequency in the first and second hearing test signals).

In some embodiments, the modulated/unmodulated method is implemented by encoding the head orientation of the infant. For example, if the infant notices that he/she has modulated the testing frequency, then the infant may hold his/her head more steadily, may rotate his/her head to the source of the hearing test signal, and/or may incline his/her head in an automatic gesture of increased interest.

Among other advantages, the methods and systems described herein provide a technique in the early detection of hearing loss in infants that is not only acceptable to physicians, but also sufficiently simple to use by parents at home. While the methods and systems focus on audiological testing, they can also be used by other specialists in other fields (e.g., experimental psychologists, speech pathologists, other clinicians, etc.) to quantitatively record and analyze an infant's behavioral responses.

The various illustrative modules and logical blocks described in connection with the embodiments disclosed herein can be implemented or performed by a machine, such as a general-purpose processor, a DSP, an ASIC, a field programmable gate array (FPGA) or other programmable logic device, discrete gate or transistor logic, discrete hardware components, or any combination thereof designed to perform the functions described herein.

The steps of a method, process, or algorithm described in connection with the embodiments disclosed herein can be embodied directly in hardware, in a software module executed by a processor, or in a combination of the two. A software module can reside in RAM memory, flash memory, ROM memory, EEPROM memory, registers, hard disk, a removable disk, or any other form of computer-readable storage medium known in the art. An exemplary storage medium can be coupled to the processor such that the processor can read information from, and write information to, the storage medium. Alternatively, the storage medium can be integral to the processor.

While the embodiments of this disclosure have been described as having exemplary designs, the embodiments can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.”

Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

Systems, methods and devices are provided herein. In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic with the benefit of this disclosure in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A device comprising:
a mouthpiece with a portion adapted to be insertable into a mouth of an infant;
a pressure sensor configured to detect pressures induced by the infant's sucking actions on the mouthpiece; and
a controller coupled to the pressure sensor, the controller being configured to transmit data associated with the detected pressures for use in assessing the infant's hearing.

2. The device of claim 1, wherein the controller is configured to transmit, wirelessly to a second device, the data associated with the detected pressures.

3. The device of claim 1, further comprising a motion sensor configured to detect head movements of the infant;
the controller further coupled to the motion sensor and configured to transmit data associated with the detected head movements for use in assessing the infant's hearing.

4. The device of claim 3, wherein the motion sensor is further configured to detect sucking motions of the infant;
the controller further configured to transmit data associated with the detected sucking motions for use in assessing the infant's hearing.

5. The device of claim 4, wherein the controller is configured to transmit, wirelessly to a second device, the data associated with the detected pressures, the data associated with the detected head movements, and the data associated with the detected sucking motions.

6. The device of claim 1, further comprising a base portion coupled to the mouthpiece, the base portion including the pressure sensor, the controller, and a motion sensor.

7. A method in a first device, the method comprising:
detecting, by a pressure sensor in the first device, pressures induced by an infant's sucking actions on a mouthpiece of the first device, the mouthpiece having a portion adapted to be insertable into a mouth of the infant; and
transmitting, from the first device for reception by a second device, data associated with the detected pressures for use in assessing the infant's hearing.

8. The method of claim 7, wherein the transmitting includes wirelessly transmitting the data associated with the detected pressures.

9. The method of claim 7, further comprising:
detecting, by the first device, head movements of the infant; and
wherein the transmitting further includes transmitting data associated with the detected head movements.

10. The method of claim 9, further comprising:
detecting, by the first device, sucking motions of the infant; and
wherein the transmitting further includes transmitting data associated with the sucking motions.

11. The method of claim 10, wherein the transmitting includes wirelessly transmitting the data associated with the detected pressures, the data associated with the detected head movements, and the data associated with the detected sucking motions.

12. A method in a second device, the method comprising:
receiving, by the second device from a first device, data associated with detected pressures induced by an infant's sucking actions on a mouthpiece in the first device; and
assessing, by the second device, the infant's hearing based on the data associated with the detected pressures.

13. The method of claim 12, wherein assessing the infant's hearing includes generating a hearing test signal with a modulated frequency that is based on the data associated with the detected pressures.

14. The method of claim 12, wherein assessing the infant's hearing includes:
generating a first hearing test signal with a frequency that is unmodulated;
generating a second hearing test signal with a modulated frequency that is based on the data associated with the detected pressures; and
determining whether a sucking behavior of the infant during the first hearing test signal is different from a sucking behavior of the infant during the second hearing test signal.

15. The method of claim 14, further comprising:
receiving, by the second device from the first device, data associated with detected head movements of the infant; and
determining whether a head movement of the infant during the first hearing test signal is different from a head movement of the infant during the second hearing test signal.

16. The method of claim 14, further comprising:
receiving, by the second device from the first device, data associated with detected sucking motions of the infant; and
determining whether a sucking motion of the infant during the first hearing test signal is different from a sucking motion the infant during the second hearing test signal.

17. The method of claim 14, wherein when the sucking behavior of the infant during the first hearing test signal is not different from the sucking behavior of the infant during the second hearing test signal, increasing intensity levels of the first and second hearing test signals until the sucking behavior of the infant during the first hearing test signal differs from the sucking behavior of the infant during the second hearing test signal.

18. The method of claim 14, wherein when the sucking behavior of the infant during the first hearing test signal is different from the sucking behavior of the infant during the second hearing test signal, decreasing intensity levels of the first and second hearing test signals to determine a minimum intensity level that will cause the sucking behavior of the infant during the first hearing test signal to differ from the sucking behavior of the infant during the second hearing test signal.

19. The method of claim 14, wherein intensity levels of the first and second hearing test signals are generated based on an ambient noise level.

20. The method of claim 19, further comprising performing a calibration procedure to ensure that the generated intensity levels of the first and second hearing test signals conform to desired intensity levels.
